SEQUENCE LISTING (16 sequences)

SEQ ID NO: 1 - DNA, 1707 bp, Artificial Sequence
  Nucleotide sequence of pdc (pyruvate decarboxylase) gene
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgccgctgaagcgatttacaccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggcacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcattcgcttccccagcgtccatctgaaagactatctgacccgtttggctcagaaagtttccaagaaaaccggtgcattggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 2 - DNA, 996 bp, Artificial Sequence
  Nucleotide sequence of ldhA (lactate dehydrogenase) gene
atgcgcgtcgcaatattcagttccaaaaactatgaccatcattctattgaaaaagaaaatgaacattatggccatgaccttgtttttctgaatgagcggcttaccaaagagacagcagaaaaagccaaagacgcagaagctgtttgtatctttgtgaatgacgaagccaatgccgaagtgctggaaattttggcaggcttaggcatcaagttggttgctcttcgttgcgccggttataacaatgtcgatctcgatgcggccaaaaagctgaatatcaaggttgtgcgcgtgcctgcctattcgccctattcggttgccgaatatgcagtagggatgttgctcaccctgaatcggcaaatttcacgcggtttgaagcgggttcgggaaaataacttctccttggaaggtttgattggccttgatgtgcatgacaaaacagtcggcattatcggtgttggtcatatcgggagcgtctttgcccatattatgacccatggttttggtgccaatgttatcgcctataaaccgcatccagaccccgaattggcgaaaaaggtcggtttccgcttcacctctctcgatgaagtgatcgagaccagcgacatcatttcgcttcactgtccgctcacgccagaaaatcatcacatgattaatgaagaaacactggcaagggcaaaaaaaggcttttacctcgtcaataccagtcgcggcggcttggttgataccaaggcggtgattaaatcgctgaaagccaaacatctcggcggttatgcggcggatgtttacgaagaggaggggcctttattcttcgaaaatcacgctgacgatattatcgaagatgatattctcgaaaggttgatcgctttcccgaatgtggttttcacgggacatcaggcctttttgacgaaagaggccttatcaaacattgctcacagtattctacaagatatcagcgatgccgaagctggaaaagaaatgccggatgcgcttgtttag

SEQ ID NO: 3 - DNA, 2933 bp, Artificial Sequence
  Nucleotide sequence of upstream homologous region of pdc gene
cctgaatagctggatatggagcccgtcaaagcgaaataagccattcgcaatattgatccatatcaagccatccgaatcctgcataattgcattgaccgttgtttgcggcagattcgggatagcacccatgtcggtaaaaaaagcagactggaaattctgccaaccatgcatagaatcgataacaccgggcaagtgaaaggttttttctgcccgacatttgacgtcttcattcgccgaacgcgaagatatgccttcccaatgagacaatagcgggcgaccattcgcaagcattgcctgaactggtattcctaaaaaaagacaccataaaaatatcggtaataaaaaccgatagaattttggccttttcgatttgcccgatattctcatgttgaacagtgtcatgtaataaaaaactaaactaacattctatagaaagaaatttatcataaatattttaaaaaaataattccttaaaaataaaatatagataaaaaattgcgggtttatttgatagttttatcgcctgttttcctaaaaataaaaataaaatttgtcaaaattattccacctaatatgattagtagatttattctattttgttaaattttgtaacttttattatttttagatagcgcggctaaagccttgttttttctgcttacgataactgtcgaacaccgccgcgacgatacgaacaaaggggcgtcctgcatcagtcatctctattcggttctgctgaaactggatcaatccttcttcttccagatgactcagttctcttttctcatcttcaagagataggcttttatcataaggcgttaaatcaaccgcaaaatgacacataagggcgctaatgatttcacctctaagatgatcttcttgcgatatatcgatacctctaaaagacgctaaaccctgttcttcaatagctcggctataatttttaatatcggcgatattctggatataggcatcggaaaaagtcgagatggccgatgcccctaaaccgatcaataccggctcgttatcggtggtataaccttgaaaattgcggcgtagcttcttttcccgtgcagctacggccaataaatcatcggttttagcgaaatgatcaataccgattgcttgatatccattttgttgcaatatttcggcaatcaaagccgcctgctcaaaacgctctttggtttggggcagggcgctttcatcaatcaatttctgattggctttccgttcgggcaaatgggcatagccaaaacaggcaattctgtcaggcgacaggcgcagcgtttcttgacaattaagacgtatctcatccaacccttgatggggcaggccatagagaaggtcaaaattaacctgactgatgcccgcttggcgcaaggccagaatagctttttcaacctgctcgatgggttgaacgcggccgatcgctttttgaatatgcgggttaaagtcttgaaccccaagactggtgcgattgatgccaatatcaaaaagattttgagcgaattcggcatccagtaaacgcgggtcaagttccatcgcatggatcatatcctcggccgcatcaaaatagcgttgcaaggccttcattaccctacggataccttcaattccgatgatggacggggttccccccccccaagagagatgacctatttttagatgattaggtgccagattaccgacagtctctatctctttaaccagagtatcaacataggctgtaatgatttcctgacggcggacaaccttggtgtgacaaccacaataatggcaaagctgctgacaaaaggggacatgaaggtaaatcgatattcgactatccggtgcaacatttttgattcggatcgcatgctctttgggcgtcaggtcattacgaaaatcagccgccgtcggataggaagtatagcgaggcacattacgtccggcataacggtcaataaaattttgttttaaaattgtaggcggctggattgtcataaagtcacacggttccttatttcttttctatccaaactctttgcaatagtctgtaacaagatgacggcgacgatatcggatcttcgtctcttttgggtcgcgaaaaaatattaactttaatcgaaaaaaattgagtctgtttttactcgggacaagaccgcctttttttatccaaagaatatccctttcatcttctttcgaaagcgaaaaataaatactgaaaacaacggttttgaccacaagattcacgggctatccttcaaaagaagaagcccttttttatcctctcttagggcgtggttaagggttggcttgggcttaacaaattttgtttatgcacaactttgggttgacttggcgacaataaaatatcaccagaggggcagaccggttacggaaacgtttccgctttgatagctcagacggagggaaaggctttgtcagtgttgcggtataatatctgtaacagctcattgataaagccggtcgctcgcctcgggcagttttggattgatcctgccctgtcttgtttggaattgatgaggccgttcatgacaacagccggaaaaattttaaaacaggcgtcttcggctgctttaggtctcggctacgtttctacatctggttctgattcccggtttacctttttcaaggtgtcccgttcctttttcccctttttggaggttggttatgtcctataatcacttaatccagaaacgggcgtttagctttgtccatcatggttgtttatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagca

SEQ ID NO: 4 - DNA, 2873 bp, Artificial Sequence
  Nucleotide sequence of downstream homologous region of pdc gene
tttttaaataaacttagagcttaaggcgaaaagcccgtccggttttaccgggcgggctttttttatccaagacgactcaaatgatggggcaggaggcatagcgcttttttacgcgttcctttttttcctgattttccctctcaacaaaaatgtcttattgaaacataaatgacagctttgtttttcttttctataaaattgttacagaatcccttgctaagcagggctggctcttgaccgataaaaagaaaagccataaagacttcttcttcgtaagggctgaattattgaagtcagtgccttaactatttattttctgattacatcgaggacaagcatggcgaatacgccgcaggcaaaaaagcgcatccgtcgtaacgaccgtcgcgctgaaattaacggtaaccgcgtcaaccgcattcgtactttcattaaaaaagtcgaatctgctattgctgctggtaacaaatccgaagctgaaaccgctttagcaagcgcacagccggaattattccgtggcgtttctaaaggtgttttgcataaaaacacggcttcacggaaattctctcgtttggcaaaaggcgtcgctgcgctcgcttaaacgtcagggaaagggatgccatcgctcttctttagaaagatggcatattccctctatttctgttccaaaccattccagccctgcttgatttgtcgtaatcaggtaaaatatatccattcgtcctgaatggatgtttttctgtatccgctttaaaaatctgacacgaccggatatgctggccgtgaaggcttgcggctgccagaaatacttttcttgaacgcgccaatagctgcgattaaaaaccgtcatttccaaggggtagattgggtaattttctctggcctatgcccgacagctataatacaggattcctgacagaatttctgtggcggtttattcaagtgattgtatcttatgaagtgattcatgtgcatatatcgagtcaagatataaatttttaagactcttgtcgcttcccccttgattggcggggattgctcttccaaaaagcaagaacataccggatcatatttgagtctgttatttaggcttcaatagacaaagaatcagatatgtcgggtctccgcgacaagagactcaagggcgcaaaagctgtcttttcaaaagatacagtttgccggaaatacactctccattgatacaaggcaaagcaaagcggggtagtagaatagcgtgctgaatccagtgcaaaaagaaggtggagatatgcagcccccgtcacaggactgggcttcattactgccagctgcatggagcgaagcgcgccagatattgcgcaaaaaatgcggcagccggacttttgaaagctggcttaaatccctgatgcttgccgattttgatagccagaaaaaaatcattcgcttggcttgccccagtgaatttatggctaactggatatcctctcatctgtcagatgagcttttgctggcatggcggacagtctggccgggcattgccgaagtcaaggtcagcgtccgtaacccagaatcgcaacctttgctcctcgatgttaccgaaatagaattaccgctaggggatcaacctcgcccattgccgaaaaaaccggcaaagaaaaaacaatctgttcccgccacgcccaaaagcactagccctgaaaaaaaggcggagggtgaggatcaaaatcaattcgaagaacgctataatttcgacaattttgttgtcggtaaagcgaatgatctagcctatcgggcggcgtgcacttttgccgaaggtggcaaactcgatttcaatccgctttttctctatggcggaacggggcttggcaaaacccatctcatgcatgccgtgggcattgaatatctcaaacggcatccgaacagcacggctttgtatatgtcggctgaaaaattcatgtatgattttgtcgcttcgatgcgcgccaaggatacccatagcttcaaagcccgcctgcgttctgccgatcttctgatgattgatgatgtccaattcattgccggaaaggattccacacaggaagaattctttcacacaatgaacgaggtcatcaccgctggccgtcgtctggttatttctgcggatcgcagcccacaagatcttgaacggattgaaagccgtattctatcccgtttgtcatggggcttggtggctgatgtcaatccggccgatttcgagttgagattaaatattatcctcaaaaaactcgaagctatgccacaggtctcaatgccagaagatatcgttttctttctggccaaacggatttgtaccaatgtgcgggaattggaaggcgcgcttaatcgggtggttgcctatgcaacgctttccaatcgccccatcaatatggatttcgttaccgaaaccttggccgatctccttcggacaacgcaacagcgcgtaacggttgaagatattcaaaaacgggtttgcgaccattatcatcttaaattagcggatatgagttctaaaagacgggacagggttattgctcgcccacgccaagttgcgatgtatctttcaaagcaactcacttcccgttcattaccagaaatcgggcaacgttttggtgggcgcgatcacaccacggttattcatgccattcggcagatagaaaagctgcggataacggatgaagatgtcgattcggatgtccgtttgctgatgcgccaatttgaaggctaaccgctttttttagaataagggttgaatgcctcgcttgtgatttttatgaatcactttctttcctattcccgaccaaccatccgaattgagaattatgcttagtttttcggctattactgtccggcttggaggccaacttatccttgatcag

SEQ ID NO: 5 - DNA, 4879 bp, Artificial Sequence
  Nucleotide sequence of upstream homologous region of ldh gene
tggcagtcctccattcagatcgaaggtgccagccttgacgggaaagaattgccagtcaaagagctcccgacattacagccgccacgctgtgtcaaaaataccgaaaacaaaccgatgaccttctttgcctattcggaagaccccgaatgtcagactttatcgcttaatgccggaaaagggcaggtgactatgtcgggtgtttgtcagaaaaatggtaaaaatctgacaaccattgaagccaaaggcgactatcacctccgcgattattctatggcctatattatgcgaagtgaagaaaacggccataaattagaagtccgtggacatatgtctgggcattctatcggcttatgccctgccaaagatagcaatgccgatgatatcaccttggggaacaaaaaccactaatacgggtgttcgtttggcgataagactatttagcaatcataggcttccgtcataaccgctaaaatcatggcagggatcgaccttacggacttcttccatgcctttctttgatagcaaggcattgtaggggcatatcggggcaaaatatatcccgatcacaaggggcgacctttgtaggacagatatgcctaaaactctaatgacgacaggcgtaaaatcctaaagactttccgtttccgatatctgatttatcattttttctgatgagtagttgctgcgtccaattcgctgatttcaggattggagtcaggtcagggtataaccctataatgatagctacgggatagaacgcttatcatcagcttctatctttgtggcagattcctgtcgcagggaaagatagtgcagaagctatcttgaccatatccctgtcatgatagcaccccgttccagatgccagatgccagatgccagatgccagatgccagatgccagatgccagatgccagatgccagatgccagatgccagatgccagatgccagatgctcgaattatatctggcaaaaccctctcacagaaaaagggcatcacccctcaatcgcagacccgaaatagatctgacgcagacattaaaaaaggccaatggaaacccattaggccttttttattctatcaggaaaggatccggttaaaaccggatacccttattaaccctgacgggctttaaagcggcgaaccgttttattgatgatataagtgcggccacgccggcgaatcacccgattgtcgcgatggcgcccctttaaggatttgagcgagttgcggattttcatggccggactacctgccttcatcttaaaaattgggaaagaaaatgaggcctctgcctatgcgctgaattattataagtcaaggaaaattctgatatatcggaaatatccttgaaagaaaaatccgatatctcttcatcaaaacagccaaagaccattggaatacaggcttttttcgttacttttttcacgattacaaaaaaggctataattcttttttgaaatattatgatatccgttaaagatcatagccacagcaaccacccttcataaccggatggcttgacggagactatcttgaggcatctcatcacgatatcacagcccgcaataccgcaagacgtgaccaataaagacggtcacaaggcaaaaaaacagctccggtgcaaaggcatctatccctttttatggatgtttttctttgcggcggcgcttccgttacaggcgacccagccgatagaagtcactcgctttcacaaaagcgatatggctacaaccggcatcgtcaacattatgcctcatgacccgaccttacggaatacgctggaatatcagcgctacacggccagcatcgcccgcaatctcacaagaatcggtttccaagtcacggacaacccgcaacaagccgaatatacgatgatgtatgacgtgatgcggggaacgcattacagagacaacggccaaacgcccccgcgtgatactcgccctcatggtggcatcagccttggcggtggttatggtggcggaggcggctttggaggcggcggtgtcggctggggcggcggcggaagtggtatcagtatcggaggcggtggcgggggtggccgcggcttcggaggtggcggaggcggtatcagtgccggtatttctgtccctgtcggtaacggctatcataccagcaacaaggtcgaaaccattctaacggcacaactcagccgcagggatacgcatcaggctatctgggaaggccgcgcccgaacggaagctaaaagtaacaaagccgaaagcacgcccgatattgcggtggacagattagccacagccatgttcggccagtttcccggtgaatcaggtgaaacggaaaaagtaaaatgacccttcaaatcaatgccgcctttgatggcggaaatatccatgttgtcgaacaagacggaaaccggatttatctggaaattatcaaagataaccagtcggatttcttccaatggttctatttcaaggtaaccggtgccaaagatcaggccttggaactggttgtcaccaatgccagcgattccgcctatccggccggctggcctgattatcaggctcgcgtttccgaagaccgccaagactggcaaatgacagaaacggattatcgcgacgggatgctgaccatccgttatacgccgcgtagtaatatcgcttattttgcctatttcgccccttactcaatggaacggcaccatgatctgattgcccgtatggctggcaagtcaggggtcggttacgaaatgttgggtaaaagcctcgatggtcaaagcatggattgcctgacgatgggggaagggcggcgctctatctggttgatcgcacggcaacatccgggcgaaaccatggccgaatggtggatggaaggcgctttggaaaggttaaccgatgaaaatgactcggttgcgcgcctgcttcgccaaaaagcccgctttcatatcatgcctaatatgaatccggacggttcttgccgtggtcatttgcggacgaatgcttgtggtgccaatctcaatcgtgaatgggcagaacccacggctgaacgcagccccgaagtgttggacgttcgcaatcatatggacaaaacgggcgttgattttgtcatggatgttcatggcgatgaagctattccgcatgtattccttgccggttttgaaggtatccccgatctcgacaaggcacaggataaattattccgccgctaccggaataaattggccaaatacacgcccgattttcaacgtcattacggttatgaaaatgacgagccggggcaggccaatctagccttggcgactaaccaattagcctatcgttacaaggcggtttcgatgacgcttgaaatgcctttcaaagatcatgacgatatgcctgatttgaaaaaaggttggtcaccggaaaggtcaaaacaattaggccgcgattgtctcgctatcttggctgaaatgattgatcagctcccgatctctggcaaagatctcgcgtaataaaactatcaggcgcaatcgtaattttgcgtctgatagagcttttcataaaggctataaccgctattgccaaaagccataggcctgcataatctgacggcgaataattttcctgaaagattggcggccatttttgctgaccgcacagattgtcagcgttaattatacatggcttcttttgttgattcgggaactgcaagcgtttaccggaacaacacataacgaagagatattgaaaaggagtggaatatgcccacgctcgttttgtcccgtcacggacagtccgaatggaaccttgaaaaccgtttcaccggttggtgggatgttaacctgactgaacagggtgttcaggaagcaacggccggtggtaaagctctggctgaaaagggttttgaattcgatatcgctttcaccagcgttctgacccgcgccatcaaaaccaccaatcttattctcgaagccggtaaaaccctttgggttccgaccgaaaaagattggcgtttgaatgaacgtcactatggtggtctgaccggtctgaacaaggctgaaaccgccgctaaacatggtgaagaacaggttcatatttggcgccgttcttatgacgttccgccgcccccgatggaaaaaggcagcaagttcgatctgtctggcgatcgccgttatgatggtgtcaagattcctgaaacggaaagcctgaaagacaccgttgctcgcgtgctgccttattgggaagaacgcattgcccctgaactgaaggctggcaagcgcgtcctgatcggtgcgcatggtaactcactgcgcgctctcgttaagcatctgtcgaaattgtcggacgaagaaatcgtcaaattcgaattgcccaccggtcagccgttggtctacgaattgaatgatgatctgactccgaaagatcgttacttccttaacgaacgttaatagccttgggcttttaaagccttttggtttgttaaccgttttttcggccagagttttctctggccgaaaatttatgtctatccctttgtttttctatccccatcacctcggttttgttgacaaaaaaaggtggccactaaattggctttccgcaccgatgggatgatttttattctttgctattcttcgctctttgcccaattcattaaaagcggaaatcatcaccaaagatagaagacgcagccttcaccatttcagattgcccttctcgggcattttctgctgctagaatcctcttaaaaatattaaattccactctattggtaatatgtttccctctttagggaacaaataaagcccttctttgttctataaaagttagcttaccgattttacaaaaaataataccgcttcattcaatcggtaatacatatcttttttcttcaaaaaacttttcaagagggtgtct

SEQ ID NO: 6 - DNA, 4984 bp, Artificial Sequence
  Nucleotide sequence of downtream homologous region of ldh gene
tagacaagcgacaattaaccttttgaagatcataatgatcaaatttttgggttaattcggtagttatggcataggctattacgcgctaattgatatcaaaaaaaagcatagccggacatcataccggctatgttttttattaggaaaaaatttcctttcaccttgcttagccatcgccgcattatttaatcaatatgccgagtttttcttgaaatccctatcttacaccaaggccaacaagggaatcatccatactcggtgtcctatcctatgactttttaaattttctccaaatttactaaaatcacgccatctcagcggctgctattttcaaaaagcgcctctcaaaaccgctttttcctgctcaaatatcggatcccaaaattccctcaaaaaaggcagggtattttttacaaaatcgcccctaatatctctcaatccgctgccttgttcatatgtttttgcaaatgatttttattaaacttttttaggcgtatttttatcaagaaaatttaaataatcacatttttattattttagatttaagtattgatacaagtgatatctataaatgtttttataactttctggatcgtaatcggctggcaatcgttttccctatattcgcaagatgtatgtcagccgcagatttgtcgactgacctctatctctccgagatatatcaacaaaaggtagtcaccatgaaagcagccgtcataactaaagatcatacgatcgaagtgaaagacaccaaattacgccctctgaaatacggggaagcgcttttggaaatggaatattgcggggtatgtcataccgatctccacgtgaaaaacggggattttggcgatgaaaccggcagaattaccggccatgaaggcatcggtatcgtcaagcaggtcggggaaggggttacttctctgaaagtcggtgaccgtgccagtgttgcatggttcttcaaaggctgcggccattgcgaatattgtgtcagtggaaatgaaacgctttgccgcaacgttgaaaatgccggttatacggttgacggcgctatggcagaagaatgcatcgtcgttgccgattactcggtcaaagtgccagatggtcttgatcctgcggttgccagcagcatcacttgcgcgggtgtaaccacctataaagcagtcaaagtttctcagatacagccgggacaatggctggctatctatggcttgggcggtttaggcaatctagcccttcaatatgccaagaatgttttcaacgccaaagtgatcgcgatcgatgtcaatgatgaacagctcgcttttgccaaagagctgggcgcagatatggtcatcaatccgaaaaacgaagatgctgccaaaatcattcaggaaaaagtcggcggcgcacatgcgacggtggtgacagctgttgccaaatccgcctttaactcggctgttgaggctatccgcgcgggtggccgtgttgtcgccgttggtctgcctcctgaaaaaatggatttgagcattcctcgcttggtgcttgacggtatcgaagtcttaggttctttggtcggaacgcgggaagatttgaaagaagccttccagtttgcagccgaaggtaaggtcaaaccgaaagtcaccaagcgtaaagtcgaagaaatcaaccaaatctttgacgaaatggaacatggtaaattcacaggccgtatggttgttgattttacccatcactaggtttccgtgaaggcggaagcataaacggaaaaagcctttctcttaccagaaaggctttttctttgtcgtctgataaaaattttcatacagaatttaatacagcaatcggtgctataagccgctatccaagcttttttcttctcatgccttctattcggcaatcgctatttaaaggctgtttttatggggcattcgccctatatataaggatattagcgtttatatataatagaaggaaatctggccttgggtgaaacaaccctccaagcagcgccccatgcccatattcaacatagcggctccgatttattggaagcggccaaggcggctttattgaaatcgggtgagcaatggacagccatgcgtgcctccgtttacaaagccttggcacaaaccaacaagccaagttcagcctatgatattgccgatattgtctctcaatccgaaggacgcagagtagctgccaacagcgtttatcgcatcctcgacatcttcgtcagtagcaatctcgcgcatcgggtcgaaagcgctaacgcctatatcgtcaacgcccatcctgaatgtcgtcatgactgcctttttctcgtctgcgaccaatgtgggggtgtgattcatatagatgatgacaagatcagccgctttttaaaagaatcggcagaaaaaaacgattttgttgcagaaaggtctgttttagaaatacggggtaaatgttcacattgtctttcccattaacctaaatgtacctcaggttaacctgttgcaatgactctattacctgctatgattttgtaacttttatgtcgcagtcagggcttatcttggctaatttgggttcctgctgttcacctttagggcgaattgttttactaaacaggcttaaatttcggtttgatttaaggccctaagcttatgtttccgaatgacaagacgccgctgttagacaagatcaagacaccggcagaattgcgtcaattagatcgcaacagcctccggcaattggcggatgaattacggaaagagaccatctcggcagtgggtgtgaccggcggacatctcggttccggtctgggggttatcgaattaacggtagcccttcactatgttttcaacacgcccaaagacgctttagtctgggatgttgggcatcaaacctatcctcacaagattttaacaggtcgccgcgatcgtattcggacattgcggcaacgtgacggcttatcgggctttacgcagcgcgcggagagcgaatatgacgcttttggagccgcgcatagttcgacttctatttctgcggcgctcggctttgcgatggccagcaaattatccgacagcgacgacaaagcggttgcgattatcggtgatggctcgatgacggcaggcatggcttatgaagccatgaataacgccaaggcggcgggtaagcgcctgattgtcattttgaatgacaatgaaatgtcgatttcaccgccggtgggtgccttatcgtcttatttgagccgcctgatttcctcacggcctttcatgaatttgcgcgatatcatgcgcggcgttgttaaccggatgccaaaaggcttggcaacggctgcccgcaaggctgatgaatatgcgcgtggtatggcaaccggtggcaccttctttgaagagctgggcttttactatgttggccccgtggatggtcataatttagatcagctcattccagttttagaaaatgtccgcgatgccaaggacggccccattttggtgcatgtcgtcactcgcaaaggccaaggctatgctccggctgaagcggccaaggacaaatatcacgccgtgcagcgcttggatgtggtttccggtaagcaggcgaaagcgcccccgggacctcccagctatacctctgttttttcggaacagctgatcaaggaagctaagcaagacgataagattgtgaccattacggcagctatgccgactggcaccggtcttgatcgttttcagcaatattttcctgaaagaatgtttgatgtcggtattgccgaacaacatgccgtaacctttgcggctggtttggcggctgccggttacaagcctttctgttgtctctattcgaccttcttgcagcgcggctatgaccagttggtgcatgatgtcgctatccagaatttgccggtgcgcttcgccgtcgatcgtgcgggtcttgtcggtgccgatggggcaacccatgcgggtagcttcgacctcgcctttatggttaatctcccgaatatggtcgtgatggcgccttccgatgaacgggaattggccaatatggtgcatagcatggcgcattatgaccaaggcccgatctcggtgcgttatccgcgtggtaatggtgtgggtgtctccttggaaggtgaaaaggaaattctgcctatcgggaaaggtcgcctgatccgtcgcggtaaaaaggttgctatcctatctctcggcactcgattggaagaatccttgaaggctgctgatcggcttgatgctcaaggtttgtcgacatcggttgctgatatgcgttttgctaagcccttggatgaagcgctgacccgccaacttttgaaaagccatcaggtcattattaccattgaagaaggcgctttgggtggttttgcaacccaagtcctgacgatggcttcggatgaaggcctgatggatgacggattgaaaatccgcaccctgcgtctgccggatcggttccagccgcaagacaagcaagaacggcaatatgccgaagccggtcttgatgctgatggcatcgttgctgcggttatctccgcattgcatcgtaattctaaacccgtggaagtcgtcgaaatggcgaatatgggtagcatcgctcgcgcttaatttgctattagggagcctcggctcccgacaatagtaaagagatcatatataatgctacatccggttgttttgtgtggtggttcaggtacacgtcttttcccgttatcccgccggagccatcccaaacaactcctcagcttgatgggcgaaaatagcctgtttcaggacgctgtcgcacgtgtaacagattcttctctattcacggcacctcttgttatctgcaatgaagaataccgttttactattgcagaacagttgcaggaaatgggcgttaaggctcaagagattgtccttgagccagaaggccggaacacagcgcccgcgattgctttagcggcgtcaatgattgcagataaagatcctgatgcctgcatgctgatcttaccgtcagatcacgttatccgggatgt

SEQ ID NO: 7 - DNA, 32 bp, Artificial Sequence
  nucleotide sequence of primer pdcF
cctgaatagctggatctagagcccgtcaaagc

SEQ ID NO: 8 - DNA, 28 bp, Artificial Sequence
  Nucleotide sequene of primer pdcR
ctgatcaaggagagctcggcctccaagc

SEQ ID NO: 9 - DNA, 26 bp, Artificial Sequence
  Nucleotide sequence of primer pr-pdcF
gagggaaaggctttgtcagtgttgcg

SEQ ID NO: 10 - DNA, 27 bp, Artificial Sequence
  Nucleotide sequence of primer dn-pdcR
tgacgcggttaccgttaatttcagcgc

SEQ ID NO: 11 - DNA, 29 bp, Artificial Sequence
  Nucleotide sequence of primer ldhAF
tggcagtcctccatctagatcgaaggtgc

SEQ ID NO: 12 - DNA, 29 bp, Artificial Sequence
  Nucleotide sequence of primer ldhAR
gtgatctgacggtgagctcagcatgcagg

SEQ ID NO: 13 - DNA, 36 bp, Artificial Sequence
  Nucleotide sequence of primer ldhA-PmeI-2R
aactagtttaaacaagagcgaagaatagcaaagaat

SEQ ID NO: 14 - DNA, 36 bp, Artificial Sequence
  Nucleotide sequence of primer ldhA-PmeI-2R
ctcttgtttaaactagttatggcataggctattacg

SEQ ID NO: 15 - DNA, 27 bp, Artificial Sequence
  Nucleotide sequence of primer npr-ldhAF
cagcaagttcgatctgtctggcgatcg

SEQ ID NO: 16 - DNA, 31 bp, Artificial Sequence
  Nucleotide sequence of primer dn-ldhAR
gattaaataatgcggcgatggctaagcaagg

(19) United States
(12) Patent Application Publication
(10) Pub. No.: US 2009/0162910 A1
(43) Pub. Date: Jun. 25, 2009

(54) METHOD FOR MASS PRODUCTION OF PRIMARY METABOLITES, STRAIN FOR MASS PRODUCTION OF PRIMARY METABOLITES, AND METHOD FOR PREPARATION THEREOF

(76) Inventors: Jeong-Sun Seo, Seoul (KR); Hyon-Yong Chong, Seoul (KR); Jeong-Hyun Kim, Seoul (KR); Jae-Young Kim, Chungcheongbuk-do (KR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/279,692

(22) PCT Filed: Feb. 16, 2007
(86) PCT No.: PCT/KR2007/000860

§ 371 Date: Aug. 15, 2008

(30) Foreign Application Priority Data
Feb. 16, 2006 (KR) 10-2006-0015116
Feb. 6, 2007 (KR) 10-2007-0011953

(51) Int. Cl. C12P 7/46; C12P 7/06; C12N 1/21; C12N 15/01

(52) U.S. Cl. 435/145; 435/161; 435/252.3; 435/440

(57) ABSTRACT

The present invention relates to a method for mass production of other primary metabolites by inhibiting a specific metabolite of metabolism in microorganisms, a transformant for mass production of other primary metabolites plasmid clone by modifying a specific gene relating to the metabolism, and a method for preparation thereof. The primary metabolites can contain lactate, succinate, or alcohol as ethanol, wherein each has a high industrial applicability as an environmental friendly plasmid clone biochemical material.

### CROSS REFERENCE TO RELATED APPLICATION

This application claims priority to and the benefit of Korean Patent Application Nos. 10-2006-0015116 filed on Feb. 16, 2006 and 10-2007-0011953 filed on Feb. 6, 2007, which are hereby incorporated by reference for all purposes as if fully set forth herein.

### BACKGROUND OF THE INVENTION

(a) Field of the Invention

The present invention relates to a method for mass production of other primary metabolites by inhibiting a specific metabolite of metabolism in microorganisms, a transformant for mass production of other primary metabolites by modifying a specific gene relating to the metabolism, and a method for preparation thereof. These primary metabolites can contain lactate, succinate, or alcohol as ethanol, wherein each has a high industrial applicability as an environmental friendly biochemical material.

(b) Description of the Related Art

Since the industrial revolution, mankind has accomplished remarkable growth with the development of the petrochemical industry as a basis, but indiscreet development and misappropriation have also brought many environmental problems that must be solved as soon as possible such as ecocide.

Due to climate changes caused by ecocide such as ozone layer depletion, the entire world is putting efforts into environmental protection countermeasures for preventing additional ecological destruction, such as the implementation of climate change agreements and the Kyoto Protocol. However, these environmental protection countermeasures will influence the extended development of petrochemical industries that consume much energy throughout the entire world and on the economic and social infrastructures of nations having high oil dependence.

Currently, research on substitute chemical products that can be produced from renewable resources, among them lactic acid and succinic acid, is receiving recognition for the possibility of developing useful biochemical products. Lactic acid has already been used to develop a biodegradable plastic, and upon its future commercial production, it has been reported that it will be a marketable commodity. Further, governments of advanced nations are actively leading research, and production techniques of polylactic acid (PLA) are being developed with fermentation production research of lactic acid through collaboration between Cargill and Dow companies of the United States, and production techniques using 1,3-propanediol (PDO) to produce polytrimethylene terephthalate (PTT) are being developed under collaboration between DuPont and Denocor companies. Compared to previously developed fibers, PLA has excellent efficiency in terms of moisture recovery ratio, elastic recovery ratio, flameproof and ultraviolet absorption, so PLA shows promise as a biodegradable environment-friendly polymer. The physical properties of nylon and polyester that are known as previously developed representative fibers, and PLA as an environment-friendly polymer, are denoted in the following Table 1.

TABLE 1

| Physical properties | Nylon | Polyester | Polylactic acid (PLA) |
|---|---|---|---|
| weight (g/ml) | 1.14 | 1.39 | 1.25 |
| strength (cN/tex) | 6.05 | 6.6 | 6.6 |
| moisture recovery ratio (%) | 4.1 | 0.2-0.4 | 0.4-0.6 |
| elastic recovery (when is | 89 | 65 | 93 |
| 5% tensile) | | | |
| Flameproof | middle | excellent | low |
| UV interception | low | middle | excellent |

As shown in the Table 1, PLA has equal or better physical properties when compared to the previously known fibers of nylon and polyester, indicating that PLA is to a fine material for substituting for chemically synthesized fiber products.

Succinic acid polymer is known as another useful biochemical product, and it has higher pliability than PLA. Furthermore, the U.S. Department of Energy (DOE) in 2004 selected succinic acid polymer as one of valuable chemical compounds derived from biomass for the future (NREL, 2004).

Succinic acid is a dicarboxylic acid and is known as an intermediary product of the TCA cycle, it consists of 4 carbons, and is a chemical material that exists in all plant and animal cells even though at a low concentration. Succinic acid and its derivatives have been widely used in plastics, food, medicine, and the cosmetics industry.

The usefulness of succinic acid as a monomer of a biodegradable polymer that can overcome non-biodegradable, which is a vulnerability of synthesized polymers, has increased with the development of the petrochemical industry. Because one-third of plastic that is currently used is being disposed of after only one use, significant environmental contamination problems induced by waste of the plastic are occurring, and because of environmental regulations stipulating that most of this plastic should be substituted with biodegradable material, many nations are starting to take a substantial interest in the biodegradable plastics industry. Currently, research related to polybutylene succinate as a biodegradable aliphatic polyester that is being considered as the next biodegradable polymer is actively being undertaken (Kirk-other, 1979).

However, the selling price of succinic acid is high compared to what industry is willing to pay, and its production and purification are also non-efficient. Because of these reasons, succinic acid is being produced mostly by a chemical synthesis method. Namely, the succinic acid is produced through a process in which succinic anhydride produced by hydrogenation of maleic anhydride is again hydrated. But, as previously described, due to changes of the process environment according to enforcement of rapidly changing environmental regulations, it has been necessary to develop a biological method as opposed to the chemical synthesis method as described above, and research related to production of succinic acid by a fermentation method with the development of microbe cultivation techniques and genetic engineering techniques is currently being undertaken. Particularly, the production method of succinic acid by a fermentation method has an economic advantage of being able to using inexpensive renewable resources as feedstock, and it uses environmentally friendly clean technology.

For mass-producing succinic acid by the fermentation method, it is demanded to develop a strain having high-efficiency. Most succinic acid fermentation microbes are known as aerotolerant anaerobes or facultative anaerobes. Because these anaerobic microbes receive many influences in the production of metabolites as well as in cell growth according to changes in external conditions compared to aerobic microbes, the physiological and environmental research related to succinic acid producing microbes is important. Further, optimal fermentation conditions are demanded for mass-producing succinic acid through analysis of the succinic acid producing metabolite cycle based on the research data (Cynthia et al., 1996).

On the other hand, according to investigations of the U.S. Renewable Fuel Association (RFA) in 2004, about 80 alcohol production enterprises in the U.S. produced about 3.5 billion gallons of alcohol, and Brazil, having abundant feedstock resources, produced about 4.0 billion gallons of alcohol. Most alcohol in the U.S. was used for fuel, the amount being about 3.0 billion gallons. In addition, most of the yield was produced by using corn as a feedstock. The biggest advantage of producing alcohol using corn as a feedstock is that it is an environmentally friendly process. That is, the method using this natural resource as an alcohol producing feedstock can induce small energy consumption and a small carbon dioxide occurrence when alcohol is produced. Also, because the method uses renewable energy, the method has advantages in that a separate expense and energy consumption occurring for waste disposal is small. Particularly, nations having high oil-dependence such as the Republic of Korea must certainly solve the problems.

Ethanol, as a representative alcohol, can have various uses such as for alcoholic drinks, industrial and laboratorial solvents, manufacturing denatured alcohol, medicine, manufacturing cosmetics, and substrates for organic synthesis, and thereof demand has greatly increased. Recently, ethanol has been widely used as a gasoline additive to improve knocking control of gasoline as a fuel and to reduce the carbon monoxide level of exhaust gas, and for substitutive energy. Most ethanol except drinking alcohol has been mainly produced by chemical synthesis, but due to increasing manufacturing costs according to rising oil prices, it is necessary to make an effort in substituting the chemical synthesis method with the fermentation method using microbes for the production of ethanol.

### SUMMARY OF THE INVENTION

An object of the present invention is to provide an optimized strain and condition for mass-producing primary metabolites as alcohol as ethanol, lactic acid, and succinic acid that have industrial applicability and are environmentally friendly biochemical materials, and is to provide a method for mass-producing primary metabolites using the strain and condition.

### BRIEF DESCRIPTION OF THE DRAWINGS

FIG. 1 is a diagram showing deletion processes of a pdc (pyruvate decarboxylase) gene in a Zymomonas mobilis (Z. mobilis) ZM4 strain according to Examples 1 and 3.

FIG. 2 is a diagram showing primer design for identifying pdc gene deletion of a ZM4 transformant manufactured in Example 1.

FIG. 3 shows the result of electrophoresis toward a ZM4 transformant manufactured in Example 1 and a wild-type ZM4 strain.

FIG. 4 is a diagram showing deletion processes of a idhA (lactate dehydrogenase) gene in a Zymomonas mobilis (Z. mobilis) ZM4 strain according to Examples 2 and 3.

FIG. 5 is a diagram showing primer design for identifying ldhA gene deletion of a ZM4 transformant manufactured in Example 2.

FIG. 6 shows the result of electrophoresis toward a ZM4 transformant manufactured in Example 2 and a wild-type ZM4 strain.

FIGS. 7A and 7B are graphs showing growth rate and productivity of a primary metabolite induced from a pdc gene-deleted transformant (Δpdc) compared to a wild-type ZM4 strain when cultured without a hydrogen supply, respectively.

FIGS. 8A and 8B are graphs showing growth rate (biomass: g/L) and productivity of a primary metabolite induced from a pdc gene-deleted transformant (Δpdc) compared to both pdc and ldhA gene-deleted transformant (Δpdc; ΔldhA) when cultured without a hydrogen supply, respectively.

FIGS. 9A and 9B are graphs showing growth rate (biomass: g/L) and productivity of a primary metabolite induced from a pdc gene-deleted transformant cultured with a hydrogen supply compared to the transformant cultured without a hydrogen supply, respectively.

FIGS. 10A and 10B are graphs showing growth rate (biomass: g/L) and productivity of a primary metabolite induced from both pdc and idhA gene-deleted transformant (Δpdc; ΔldhA) cultured with a hydrogen supply compared to the transformant cultured without a hydrogen supply, respectively.

FIGS. 11A to 11C are graphs showing cell growth, glucose consumption, and productivity of primary metabolites induced from a idhA gene-deleted transformant compared to a Z. mobilis ZM4 strain, respectively.

### DETAILED DESCRIPTION OF THE PREFERRED EMBODIMENTS

A more complete appreciation of the invention, and many of the attendant advantages thereof, will be readily apparent as the same becomes better understood by reference to the following detailed description.

The present invention relates to a method for mass production of other primary metabolites by inhibiting a specific metabolite of metabolism in microorganisms; a transformant for mass production of other primary metabolites by modifying a specific gene relating to the metabolism; and a method for preparation thereof. The primary metabolites can contain alcohol, lactate, or succinate having high industrial applicability as environmentally friendly biochemistry materials.

The present invention is able to use Zymomonas mobilis (Z. mobilis) as a strain for mass-producing primary metabolites. The Z. mobilis is known as an alcohol fermentation microorganism with an excellent product conversion rate compared to cell growth. Theoretically, the product yield of the Z. mobilis is more than about 98% and the ethanol productivity is up to 5 g/g/L, and in more detail, the Z. mobilis produces 2 moles of ethanol per mole of glucose having a glucose metabolic rate of more than 10 g/g/h.

In the metabolism of a Z. mobilis, the main pathway of the metabolism includes the following steps:

is conversed pyruvate produced by glycolysis with acetaldehyde; and

finally, ethanol is produced by alcohol dehydrogenase. In this way, a representative enzyme relating to high efficiency of ethanol production is pyruvate decarboxylase, and the key enzyme intermediates conversion of pyruvate with acetaldehyde. Therefore, if the production of pyruvate decarboxylase is blocked, alcohol is not produced by interrupting conversion of pyruvate with acetaldehyde, and host cells come to produce other primary metabolites except alcohol using pathways other than the alcohol producing pathway for energy production.

As kinds of these primary metabolites, there is ethanol as a C2 metabolite, lactate and pyruvate as C3 metabolites, citrate as C6, glutamate as C5 metabolites, and succinate, fumarate, and malate as C4 metabolites. Therefore, if the ethanol metabolism is inhibited as above, other primary metabolites such as lactate, pyruvate, citrate, glutamate, succinate, fumarate, and malate are increased, and particularly, the productivity of lactate and succinate is remarkably increased. In the production pathway of lactate, the succinate production can be further increased by inhibiting lactate dehydrogenase production intermediating conversion of pyruvate with lactate, and then by inhibiting lactate production.

Also, a Z. mobilis appears to use lactate as an electron donor with a previously unknown partial TCA (tricarboxylic acid) cycle, and it promotes cell growth and ethanol-producing rate through inducing further anaerobic fermentation by inhibiting the lactate production, and it further produces butanediol by changing substrate-specificity of pyruvate decarboxylase.

In this way, other primary metabolites except a primary metabolite produced by the specific metabolism can be increased by inhibiting a specific metabolism in microorganisms.

Based on this point, the present invention provides a method for mass production of other primary metabolites, particularly alcohol as ethanol, succinate, and lactate by blocking the production of pyruvate decarboxylase and/or lactate dehydrogenase in a Z. mobilis and then by inhibiting the production of alcohol and/or lactate.

In more detail, the present invention provides a method for mass-producing other primary metabolites except alcohol by deleting the pyruvate decarboxylase coding pdc gene (SEQ ID NO: 1) and/or the lactate dehydrogenase coding ldhA gene (SEQ ID NO: 2), and then by inhibiting the production of pyruvate decarboxylase and/or lactate dehydrogenase.

In the Z. mobilis strain that obtains energy by alcohol fermentation, because the pdc gene derived from the strain is an essential gene for survival, if the gene is deleted it has been predicted that the strain is not able to survive. However, preferred specific embodiment(s) of the present invention demonstrated that the pdc gene-deleted strain is able to survive even though its growth is retarded by about 2 times compared to a wild-type strain, and it is able to increase the production of other primary metabolites except alcohol, for example lactate, pyruvate, citrate, glutamate, succinate, fumarate, and malate.

That is, if the pdc gene is deleted from the Z. mobilis genome, the strain comes to have the possibility of using rapidly mass-accumulated pyruvate for mass-producing useful products because of the removed ethanol productivity, and can be developed and applied as a “Cell Factory Z. mobilis” for producing various useful products except ethanol. In this way, the useful products that are mass-produced by the strain can comprise pyruvate, glycerol, and lactic acid obtained from acetyl-coA, 3-hydroxypropionic acid, 3-hydroxybutanoic acid, 1,3-propanediol, glutamic acid, polyglutamic acid, aspartic acid, malic acid, fumaric acid, succinic acid, citric acid, adipic acid, pyruvate, glycerol, xylitol, sorbitol, and arabinitol. Also, the strain can mass-produce isoprenoid compounds such as coenzyme Q10, polyprenyl diphosphates, polyterpene, diterpene, monoterpene, triterpene, and sesquiterpene, wherein the compounds can be used as cosmetics additives, protectants, and precursors of medical drugs.

In the case of succinate, it was confirmed that the production of the succinate is increased by more than about 100%. Because the strain for mass production of a C4 metabolite, differently from known C2, C3, C5 and C6 metabolites, is little developed, and the succinate is widely used in various application fields such as the plastic and resin field, the medicine field, the cosmetics field, the agriculture field, the detergent/emulsifier field, the textile field, the photography field, the catalysis field, and the plating process field, it is very significant that the productivity improvement of succinate as a C4 metabolite according to the present invention is possible.

The metabolic pathway of a Z. mobilis can be represented by the following Reactive Formula 1:

In one aspect, the present invention relates to a method for mass-producing primary metabolites of a Z. mobilis by deleting at least one gene selected from the group consisting of the pdc gene (SEQ ID NO: 1) and the idhA gene (SEQ ID NO: 2) derived from the Z. mobilis genome. The primary metabolites can include at least one metabolite selected from the group consisting of ethanol, lactate, pyruvate, citrate, glutamate, succinate, fumarate, and malate.

In more detail, the present invention provides a method for mass-producing primary metabolites other than alcohol by deleting the pdc gene (SEQ ID NO: 1) derived from the Z. mobilis genome and then by inhibiting the alcohol-producing pathway. The primary metabolites can include at least one metabolite selected from the group consisting of lactate, pyruvate, citrate, glutamate, succinate, fumarate, and malate, and can more preferably include lactate and/or succinate.

The metabolic pathway of the pdc gene-deleted Z. mobilis can be represented by the following Reactive Formula 2:

Also, the present invention provides a method for mass-producing primary metabolites other than lactate by deleting the ldhA gene (SEQ ID NO: 2) derived from the Z. mobilis genome and then by inhibiting the lactate-producing pathway. The primary metabolites can include at least one metabolite selected from the group consisting of ethanol, pyruvate, citrate, glutarnate, succinate, fumarate, and malate, and can more preferably include ethanol and/or succinate.

The metabolic pathway of the ldhA gene-deleted Z. mobilis can be represented by the following Reactive Formula 3:

Further, the present invention provides a method for mass-producing primary metabolites other than alcohol and lactate by deleting both the pdc gene (SEQ ID NO: 1) and the ldhA gene (SEQ ID NO: 2) derived from the Z. mobilis genome and then by inhibiting both the alcohol- and lactate-producing pathways. The primary metabolites can include at least one metabolite selected from the group consisting of pyruvate, citrate, glutamate, succinate, fumarate, and malate, and can more preferably include succinate.

The metabolic pathway of both the pdc gene- and the ldhA gene-deleted Z. mobilis can be represented by the following Reactive Formula 4:

In another aspect, the present invention relates to a Z. mobilis transformant that has at least one gene selected from the group consisting of the pdc gene (SEQ ID NO: 1) and the ldhA gene (SEQ ID NO: 2) derived from the Z. mobilis genome deleted.

In more detail, the present invention provides a pdc gene- (SEQ ID NO: 1) deleted Z. mobilis transformant. The transformant can mass-produce at least one metabolite selected from the group consisting of lactate, pyruvate, citrate, glutamate, succinate, fumarate, and malate, and can more preferably mass-produce lactate and/or succinate. In the preferred specific embodiment(s) of the present invention, the pdc gene- (SEQ ID NO: 1) deleted transformant can be a KCTC 11012BP strain.

Also, the present invention provides a ldhA gene- (SEQ ID NO: 2) deleted Z. mobilis transformant. The transformant can mass-produce at least one metabolite selected from the group consisting of ethanol, pyruvate, citrate, glutamate, succinate, fumarate, and malate, and can more preferably mass-produce ethanol and/or succinate. In the preferred specific embodiment(s) of the present invention, the idhA gene- (SEQ ID NO: 2) deleted transformant can be a KCTC 11013BP strain.

Also, the present invention provides both a pdc gene- (SEQ ID NO: 1) and a idhA gene- (SEQ ID NO: 2) deleted Z. mobilis transformant. The transformant can mass-produce at least one metabolite selected from the group consisting of pyruvate, citrate, glutamate, succinate, fumarate, and malate, and can more preferably mass-produce succinate. In the preferred specific embodiment(s) of the present invention, both the ldhA gene- (SEQ ID NO: 2) and the ldhA gene (SEQ ID NO: 2) deleted transformant can be a KCTC 10908BP strain.

In a further aspect, the present invention provides a method of preparing a Z. mobilis transformant, which includes the step of deleting at least one gene selected from the group consisting of a pdc gene (SEQ ID NO: 1) and a ldhA gene (SEQ ID NO: 2) derived from the Z. mobilis genome.

In more detail, the method of preparing the pdc gene-deleted Z. mobilis transformant includes the following steps:

cloning the fragment containing the Z. mobilis pdc gene (SEQ ID NO: 1) into a plasmid;

removing the pdc gene from the pdc gene containing-plasmid; and

transforming the pdc gene-deleted plasmid into a Z. mobilis genome containing the pdc gene.

In the cloning steps, the fragment containing the Z. mobilis pdc gene can include a homologous region for homologous recombination located in both the 5′- and 3′-terminal regions of the pdc gene together with the Z. mobilis pdc gene, wherein the Z. mobilis pdc gene region can be substituted with the pdc gene-deleted region in the plasmid. The homologous region for homologous recombination can include 1,500 to 5,000 bp of polynucleotides located in both the 5′- and 3′-terminal regions of the Z. mobilis pdc gene, and more preferably, the homologous region can include both the polynucleotide containing from the 5′-terminal region of the pdc gene to upstream of the SacI region (upstream homologous region, 2,933 bp, SEQ ID NO: 3) and the polynucleotide containing from the 3′-terminal region of the pdc gene to downstream of the XbaI region (downstream homologous region, 2,873 bp, SEQ ID NO: 4).

In order to easily select the pdc gene-deleted Z. mobilis transformant, the pdc gene is removed, and then the pdc gene-deleted region can be substituted with a suitable selection-marker. The selection-marker can include a chloramphenicol-resistant gene (cm^{R}), a tetracycline-resistant gene (tet^{R}), an ampicillin-resistant gene (amp^{R}), or a kanamycin-resistant gene (km^{R}).

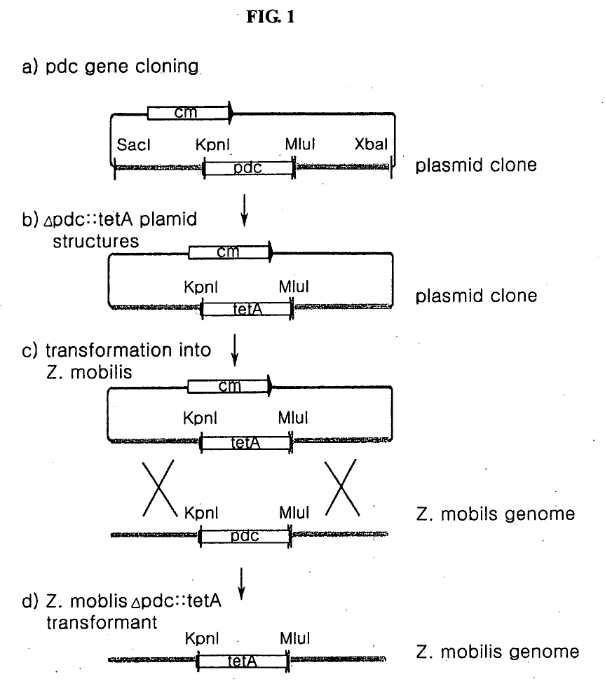

The method of preparing a pdc gene-deleted Z. mobilis transformant according to one specific embodiment(s) of the present invention is depicted in FIG. 1.

Also, the method of preparing a ldhA gene-deleted Z. mobilis transformant includes the following steps:
cloning the fragment containing the Z. mobilis ldhA gene (SEQ ID NO: 2) into a plasmid;
removing the ldhA gene from the ldhA gene-containing plasmid; and transforming the ldhA gene-deleted plasmid into a Z. mobilis genome containing the ldhA gene.

In the cloning steps, the fragment containing the Z. mobilis ldhA gene can include a homologous region for homologous recombination located in both the 5′- and 3′-terminal regions of the ldhA gene together with the Z. mobilis ldhA gene, wherein the Z. mobilis ldhA gene region can be substituted with a ldhA gene-deleted region in the plasmid. The homologous region for homologous recombination can include 1,500 to 5,000 bp of polynucleotides located in both the 5′- and 3′-terminal regions of the Z. mobilis ldhA gene, and more preferably, the homologous region can include both the polynucleotide containing from the 5′-terminal region of the pdc gene to upstream of the SacI region (upstream homologous, region, 4,879 bp, SEQ ID NO: 5) and the polynucleotide containing from the 3′-terminal region of the ldhA gene to downstream of the XbaI region (downstream homologous region, 4,894 bp, SEQ ID NO: 6).

In order to easily select the ldhA gene-deleted Z. mobilis transformant, the ldhA gene is removed, and then the ldhA gene-deleted region can be substituted with a suitable selection-marker. The selection-marker can include a chloramphenicol-resistant gene (cm^{R}), a tetracycline-resistant gene (tet^{R}), an ampicillin-resistant gene (amp^{R}), or a kanamycin-resistant gene (km^{R}).

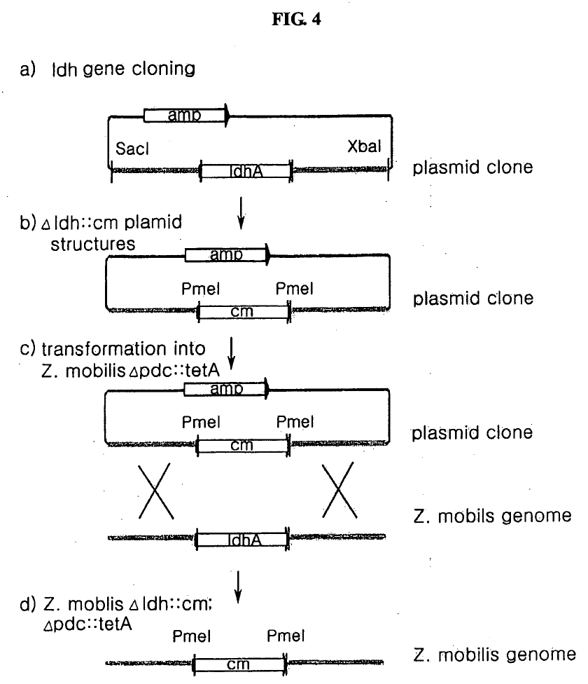

The method of preparing a ldhA gene-deleted Z. mobilis transformant according to another specific embodiment(s) of the present invention is depicted in FIG. 4.

Also, the present invention provides a method of preparing both the pdc and the ldhA gene-deleted Z. mobilis transformant, which includes consecutive steps of:

preparing the pdc gene-deleted Z. mobilis transformant; and preparing the ldhA gene-deleted Z. mobilis transformant.

Further, the present invention provides a method for mass-producing at least one primary metabolite selected from the group consisting of ethanol, lactate, pyruvate, citrate, glutamate, succinate, fumarate, and malate by culturing the pdc gene and/or ldhA gene-deleted Z. mobilis transformant. Herein, the culture temperature and culture time are not particularly limited, preferably the temperature can be 30 to 34° C., and the culture time can be 10 to 14 h.

In the mass-producing method, the productivity of the primary metabolite can be increased by using a culture medium of the Z. mobilis transformant additionally containing carbon dioxide, because the carbon dioxide acts as a carbon source when glucose in the strain is changed with the primary metabolite. For example, the production of Z. mobilis succinate is mainly achieved by a malic enzyme, wherein the succinate is necessarily carboxylated for producing malate (C4) from pyruvate (C3), and the productivity of succinate can be increased by the carbon supply.

Herein, the carbon supply is not particularly limited, and the carbon supply can include carbon dioxide or carbonate. The carbonate can use any carbonate, and more preferably it can be selected from the group consisting of NAHCO₃, NA₂CO₃, and CaCO₃. Considering effective action with the primary metabolite of transferase in the metabolism, the carbon dioxide gas can be added to the culture medium with a 0.1 to 1 vvm (aeration volume/medium volume/minute), and carbonate can be added to the culture medium at 1 to 50 mM, and more preferably at 5 to 20 mM.

With a carbon dioxide supply, the hydrogen supply is also a very important component in the production of a primary metabolite such as succinate. Herein, the hydrogen supply improves electron transfer in the cells, and then increases production efficiency of the primary metabolite such as succinate by fumarate reductase. For example, because Z. mobilis that is known as an anaerobic microbe cannot produce ATP using NADH in the cells, the NADH (NADH+H⁺) is mostly oxidized with NAD by NADH dehydrogenase, herein produced protons (H⁺) are used to maintain ΔpH, and electrons are transferred to fumarate through an electron transfer channel such as quinone and cytochrome, succinate is finally produced by fumarate reductase. Hydrogen supplied from the outside is introduced into cells through cell-membrane existing quinone, wherein the quinone has a function of electron transfer intermediation through changing hydrogen with protons and electrons through a quinone cycle in the cell-membrane, supplying protons induced from hydrogen into the cells, and transferring electrons to cytochrome. Therefore, because the hydrogen supply into the culture medium induces identical effects with the proton (H⁺) supply produced by oxidized NADH with NAD, the production efficiency of the primary metabolite such as succinate by the electron transfer promotion can be increased. Herein, the hydrogen can be added in a culture medium under a gas condition, and more preferably the hydrogen content can be added in a culture medium at 0.2 to 1 vvm (aeration volume/medium volume/minute).

In the specific embodiment(s) of the present invention, the Z. mobilis transformant can be cultured in a RM medium (glucose 50 g/L, yeast extract 10 g/L, MgSO₄ 1 g/L, (NH₄)₂SO₄ 1 g/L, KH₂PO₄ 2 g/L, pH 5.2) containing 10 mM of NaHCO₃, or 1 vvm of carbon dioxide gas for 14 h at 30° C. As a result, the production of succinate can be further increased, and the production efficiency of succinate also can be improved to a maximum of 5 g/g/h.

The present invention is further explained in more detail with reference to the following examples. These examples, however, should not be interpreted as limiting the scope of the present invention in any manner.

### EXAMPLE

### Example 1

### Preparation of a pdc Gene-Deleted Zymomonas mobilis (Z. mobilis) Transformant

According to the method shown in FIG. 1, a pdc gene-deleted Zymomonas mobilis transformant was prepared. It will be explained with reference to FIG. 1.

1-1. Cloning of a pdc Gene

A gene fragment corresponding to 7,513 bp nucleotide sequences containing a pdc gene derived from a Zymomonas mobilis (hereinafter referred to as ‘Z. mobilis’) genome (AE008692) was gained by a polymerase chain reaction (PCR) method. The primers used in the PCR reaction are as follows.

Forward primer (pdcF):

(SEQ ID NO:7)

5′-CCTGAATAGCTGGATCTAGAGCCCGTCAAAGC-3′
Reverse primer (pdcR):

(SEQ ID NO:8)

5′-CTGATCAAGGAGAGCTCGGCCTCCAAGC-3′

The fragment obtained from PCR was cut with SacI (NEB, New England Biolab, MA, USA) and XbaI (NEB, New England Biolab, MA, USA) enzymes, and then it was sub-cloned in a open pHSG398 vector (Takara Shuzo Co., Ltd., Kyoto, Japan) treated with SacI and XbaI enzymes. As shown in step a) of FIG. 1, the fragment containing the pdc gene includes a pdc gene (1,707 bp), a polynucleotide containing from the 5′-terminal region of the pdc gene to upstream of the SacI region (upstream homologous region, 2,933 bp, SEQ ID NO: 3), and a polynucleotide containing from the 3′-terminal region of the pdc gene to downstream of the XbaI region (downstream homologous region, 2,873 bp, SEQ ID NO: 4). Both the 5′ and 3′ homologous regions are used for homologous recombination with the genome of Z. mobilis when transforming them into the Z. mobilis strain.

1-2. Construction of Plasmid where a pdc Gene was Substituted with a tet^{R} Gene (Δpdc::tet^{R})

The plasmid obtained from step 1-1) was cut with KpnI (NEB, New England Biolab, MA, USA) and MluI (NEB, New England Biolab, MA, USA) enzymes, and then a tet^{R} gene (J01749) amplified by PCR from a pBR322 vector was inserted into the plasmid. As a result, the plasmid containing the tet^{R} gene substituted for the pdc gene was prepared.

1-3. Transformation of a Z. mobilis

The plasmid obtained from step 1-2) was introduced into a Z. mobilis ZM4 (ATCC 31821) strain using electroporation. In more detail, the Z. mobilis ZM4 strain was cultured in a RM liquid medium (glucose 50 g/L, yeast extract 10 g/L, MgSO₄ 1 g/L, (NH₄)₂SO₄ 1 g/L, KH₂PO₄ 2 g/L, pH 5.2) for 10 h, and then cultured in new a RM medium for 4 h until the O.D value approached 0.3-0.4 at 600 nm. The culture medium was left in ice for 20 min, and the supernatant was removed by centrifugation at 5,000 rpm for 5 min, and then washed with 10% glycerol. After washing 3 times, the plasmid was transformed into a Z. mobilis ZM4 strain that was concentrated with 100 μl of volume. The electroporation was performed using GenePulser System (Bio-Rad Chemical Division, USA), wherein the conditions for electroporation were to 1.0 kV, 25 uF, and 400Ω, respectively, and wherein the time constant was to 8.8-9.9.

In accordance with the homologous recombination between the 5′ and 3′ homologous regions located in the plasmid and hereupon each of homologous regions on a Z. mobilis ZM4 genome when is transformed, the pdc gene on the Z. mobilis ZM4 genome was deleted, and the tet^{R} gene located in the plasmid was inserted. As a result, the Z. mobilis transformant (Δpdc::tet^{R}) where the pdc gene was substituted with the tet^{R} gene was obtained. The Z. mobilis transformant (Δpdc::tet^{R}) was deposited with the Korean Collection for Type Culture (Korea Research Institute of Bioscience and Biotechnology, Taejon, Republic of Korea) on Oct. 26, 2006, and assigned deposition No. KCTC11012BP.

1-4. Selection and Identification of a Z. mobilis Δpdc::tet^{R} Transformant

The transformant obtained from step 1-3) was cultured in a RM solid medium (ethanol 20 g/L, glucose 50 g/L, yeast extract 10 g/L, MgSO₄ 1 g/L, (NH₄)₂SO₄ 1 g/L, KH₂PO₄ 2 g/L, tetracycline 15 μg/ml, pH 5.2) containing tetracycline at 30° C. for 5 days, and then living cells were collected.

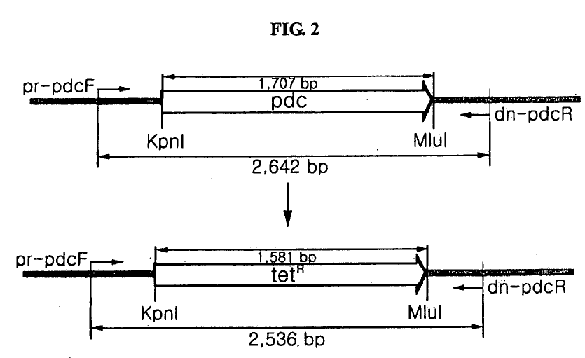

For identifying whether the collected cells were the Z. mobilis transformant (Δpdc::tet^{R}) or not, an embodiment of the present invention used the method shown in FIG. 2. As shown in FIG. 2, in the case of a wild-type Z. mobilis genome containing the pdc gene, the length of DNA sequences between the primer (pr-pdcF) region located upstream of the pdc gene and the primer (dn-pdcR) region located downstream of the pdc gene was to 2,642 bp, and on the other hand, in the case of a Z. mobilis transformant where the pdc gene was substituted with the tet^{R} gene, the length of DNA sequences between the two primers was to 2,536 bp. Therefore, by identifying the length of the region amplified by PCR using the primer set toward the genome of collected living cells, it can be evaluated whether the Z. mobilis Δpdc::tet^{R} is the transformant or not.

In more detail, the genomic DNA of the collected living cells was isolated using a DNA Easy Tissue Kit (QIAGEN Corp., Valencia, Calif., USA) according to the manufacture's instructions. Then, PCR reaction toward the genome DNA was performed using a primer set as follows.

| | |
|---|---|
| Forward primer (pr-pdcF): | |
| 5′-GAGGGAAAGGCTTTGTCAGTGTTGCG-3′ | (SEQ ID NO:9) |
| Reverse primer (dn-pdcR) | |
| 5′-TGACGCGGTTACCGTTAATTTCAGCGC-3′ | (SEQ ID NO:10) |

As a control, a wild-type Z. mobilis was treated with the two primers as above.

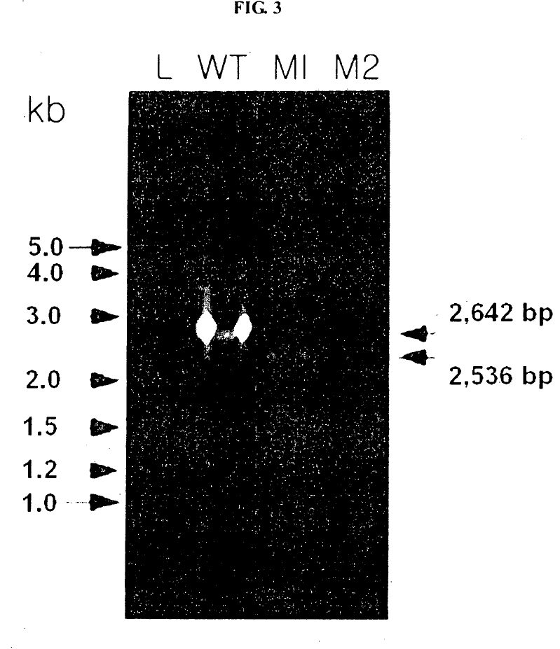

The results are shown in FIG. 3. In FIG. 3, WT indicates a wild-type Z. mobilis as a control, and M1 and M2 indicate Z. mobilis Δpdc::tet^{R} transformants, respectively. As shown in FIG. 3, an embodiment of the present invention obtained a nucleotide fragment of 2536 bp, indicating that the pdc gene was deleted.

### Example 2

### Preparation of a ldhA Gene-Deleted Z. mobilis Transformant

According to the method shown in FIG. 4, a ldhA gene-deleted Zymomonas mobilis transformant was prepared. It will be explained with reference to FIG. 4.

2-1. Cloning of a ldhA Gene

A gene fragment corresponding to 10,859 bp nucleotide sequences containing a ldhA gene derived from a Z. mobilis genome (AE008692) was gained by a polymerase chain reaction (PCR) method. The primers used in PCR reaction are as follows.

| | |
|---|---|
| Forward primer (ldhAF): | |
| 5′-TGGCAGTCCTCCATCTAGATCGAAGGTGC-3′ | (SEQ ID NO:11) |
| Reverse primer (ldhAR) | |
| 5′-GTGATCTGACGGTGAGCTCAGCATGCAGG-3′ | (SEQ ID NO:12) |

The fragment obtained from PCR was cut with SacI (NEB, New England Biolab, MA, USA) and XbaI (NEB, New England Biolab, MA, US) enzymes, and then it was sub-cloned in a open pGEM-T vector (Promega, Madison, Wis., USA) treated with SacI and XbaI enzymes. As shown in step a) of FIG. 4, the gene fragment contains a ldhA gene (996 bp), a polynucleotide containing from the 5′-terminal region of the ldhA gene to upstream of the SacI region (upstream homologous region, 4,879 bp, SEQ ID NO: 5), and a polynucleotide containing from the 3′-terminal region of the ldhA gene to downstream of the XbaI region (downstream homologous region, 4,984 bp, SEQ ID NO: 6). Both the 5′ and 3′ homologous regions are used for homologous recombination with the genome of Z. mobilis when transforming them into the Z. mobilis strain.

2-2. Construction of Plasmid where a ldhA Gene was Substituted with a cm^{R} Gene (ΔldhA::cm^{R})

For achieve the purpose, PCR reaction was performed using the plasmid obtained from step 2-1) as a template together with a primer set designed by simultaneously amplifying only idhA upstream and downstream regions. As a result, a gene fragment was obtained. The primers used in PCR reaction are as follows.

Forward primer (ldhA-PmeI-2F):

(SEQ ID NO:13)

5′-AACTAGTTTAAACAAGAGCGAAGAATAGCAAAGAAT-3′
Reverse primer (ldhA-PmeI-2R)

(SEQ ID NO:14)

5′-CTCTTGTTTAAACTAGTTATGGCATAGGCTATTACG-3′

The gene fragment was cut with PmeI (NEB, New England Biolab, MA, USA) enzyme, and then a cm^{R} gene (U08461) amplified by PCR from a pHSG398 vector (Takara Shuzo Co., Ltd., Kyoto, Japan) was inserted into the plasmid. As a result, the plasmid containing the cm^{R} gene substituted for the ldhA gene was prepared.

2-3. Transformation of a Z. mobilis

The plasmid obtained from step 2-2) was introduced into a Z. mobilis ZM4, (ATCC 31821) strain using electroporation. In more detail, the Z. mobilis ZM4 strain was cultured in a RM liquid medium (glucose 50 g/L, yeast extract 10 g/L, MgSO₄ 1 g/L, (NH₄)₂SO₄ 1 g/L, KH₂PO₄ 2 g/L, pH 5.2) for 10 h, and then cultured in new a RM medium for 4 h until the O.D value approached 0.3-0.4 at 600 nm. The culture medium was left in ice for 20 min, and the supernatant was removed by centrifugation at 5000 rpm for 5 min, and then harvested cells were washed with 10% glycerol. After washing 3 times, the plasmid was transformed into a Z. mobilis ZM4 strain that was concentrated with 100 μl of volume.

In accordance with the homologous recombination between the 5′ and 3′ homologous regions located in the plasmid and hereupon each of homologous regions on Z. mobilis ZM4 genome when is transformed, the pdc gene on the Z. mobilis ZM4 genome was deleted, and the cm^{R} gene located in the plasmid was inserted. As a result, the Z. mobilis transformant (ΔldhA::cm^{R}) where the ldhA gene was substituted with the cm^{R} gene was obtained. The Z. mobilis transformant (ΔldhA::cm^{R}) was deposited with the Korean Collection for Type Culture (Korea Research Institute of Bioscience and Biotechnology, Taejon, Republic of Korea) on Oct. 26, 2006, and assigned deposition No. KCTC11013BP

2-4. Selection and Identification of a Z. mobilis ΔldhA::cm^{R} Transformant

The transformant obtained from step 2-3) was cultured in a RM solid medium (glucose 50 g/L, yeast extract 10 g/L, MgSO₄ 1 g/L, (NH₄)₂SO₄ 1 g/L, KH₂PO₄ 2 g/L, chloramphenicol 75 μg/ml, pH 5.2) containing chloramphenicol at 30° C. for 5 days, and then chloramphenicol-resistant living cells were collected.

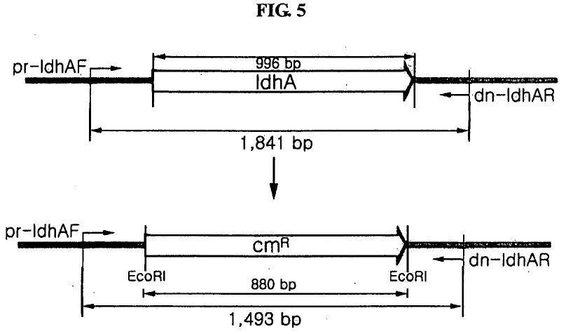

For identifying whether the collected cells were Z. mobilis transformant (ΔldhA::cm^{R}) or not, an embodiment of the present invention used the method shown in FIG. 5. The chloramphenicol-resistant living cells were cultured in a RM liquid medium (glucose 50 g/L, yeast extract 10 g/L, MgSO₄ 1 g/L, (NH₄)₂SO₄ 1 g/L, KH₂PO₄ 2 g/L, chloramphenicol 75 μg/ml, pH 5.2) at 30° C. for 16 h, and the supernatant was removed by centrifugation at 5,000 rpm for 5 min, and then the cells were collected. As shown in FIG. 5, in the case of a wild-type Z. mobilis genome containing the ldhA gene, the length of DNA sequences between the primer (pr-ldhAF) region located upstream of the ldhA gene and the primer (dn-ldhAR) region located downstream of the idhA gene was to 1,861 bp, on the other hand, in the case of the Z. mobilis transformant where the ldhA gene was substituted with the cm^{R} gene, the length of DNA sequences between the two primers was to 1,493 bp. Therefore, by identifying the length of the region amplified by PCR using the primer set toward the genome of collected living cells, it can be evaluated whether the Z. mobilis ΔldhA::cm^{R} is the transformant or not.

In more detail, the genomic DNA of the collected living cells was isolated using a DNA Easy Tissue Kit (QIAGEN Corp., Valencia, Calif., USA) according to the manufacture's instructions. Then, PCR reaction toward the genome DNA was performed using a primer set as follows.

Forward primer (npr-ldhAF):

5′-CAGCAAGTTCGATCTGTCTGGCGATCG-3′ (SEQ ID NO: 15)

Reverse primer (dn-ldhAR)

5′-GATTAAATAATGCGGCGATGGCTAAGCAAGG-3′ (SEQ ID NO: 16) As a control, a wild-type Z. mobilis was treated with the two primers as above.

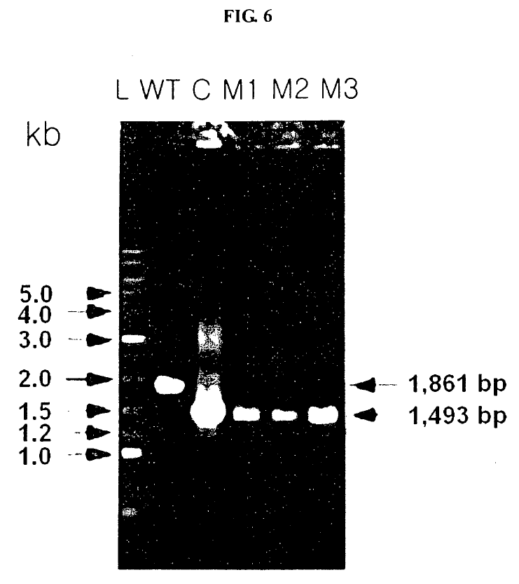

The results are shown in FIG. 6. In FIG. 6, WT indicates a wild-type Z. mobilis as a control, and M1, M2, and M3 indicate Z. mobilis ΔldhA::cm^{R} transformant, respectively. As shown in FIG. 6, an embodiment of the present invention obtained a nucleotide fragment of 1,493 bp, indicating that the ldhA gene was deleted.

### Example 3

### Preparation of Both pdc and ldhA Genes-Deleted Z. mobilis Transformant

Next, the process of Examples 1 and 2 was continuously performed, and thereafter pdc and ldhA genes-deleted Z. mobilis transformant (Δpdc::tet^{R}/ΔldhA::cm^{R}) was prepared. The Z. mobilis transformant (Δpdc::tet^{R}/ΔldhA::cm^{R}) was deposited with the Korean Collection for Type Culture (Korea Research Institute of Bioscience and Biotechnology, Taejon, Republic of Korea) on Feb. 15, 2006, and assigned deposition No. KCTC 10908BP

### Example 4

### Test for Productivity of Primary Metabolites

For investigate productivity of primary metabolites of each Z. mobilis transformant, the Z. mobilis transformants prepared from Examples 1 to 3 were used. As a control, a wild-type Z. mobilis ZM4 strain was used.

In more detail, a wild-type Z. mobilis ZM4 (ATCC 31821), a Z. mobilis Δpdc::tet^{R} transformant, a Z. mobilis ΔldhA::cm^{R} transformant, and a Z. mobilis Δpdc::tet^{R}/≢ldhA::cm^{R} transformant were cultured in a RM liquid medium (glucose 50 g/L, yeast extract 10 g/L, MgSO₄ 1 g/L, (NH₄)₂SO₄ 1 g/L, KH₂PO₄ 2 g/L, tetracycline 15 μg/ml, pH 5.2) at 30° C. for 16 h, respectively. Herein, the transformants were prepared from Examples 1 to 3. After cultivation, the cells were removed by centrifugation, and then primary metabolites obtained from the cultured supernatant were measured using HPLC (high performance liquid chromatography). In the HPLC measurement, a Hitachi HPLC System (Model D-7000, Tokyo, Japan) was used, and the metabolites were separated using an Aminex HPX-87H column (Bio-Rad, USA). Among the primary metabolites, organic acid was identified and quantified with a UV (ultraviolet) detector (Hitachi D-4200, Tokyo, Japan), sugar and ethanol with an RI (refractive index) detector (Hitachi D-3300, Tokyo, Japan), respectively. 0.0025 N of sulfuric acid was used as a mobile phase (solvent), the column temperature was to 60° C., and the flow rate was to 0.6 ml/min.

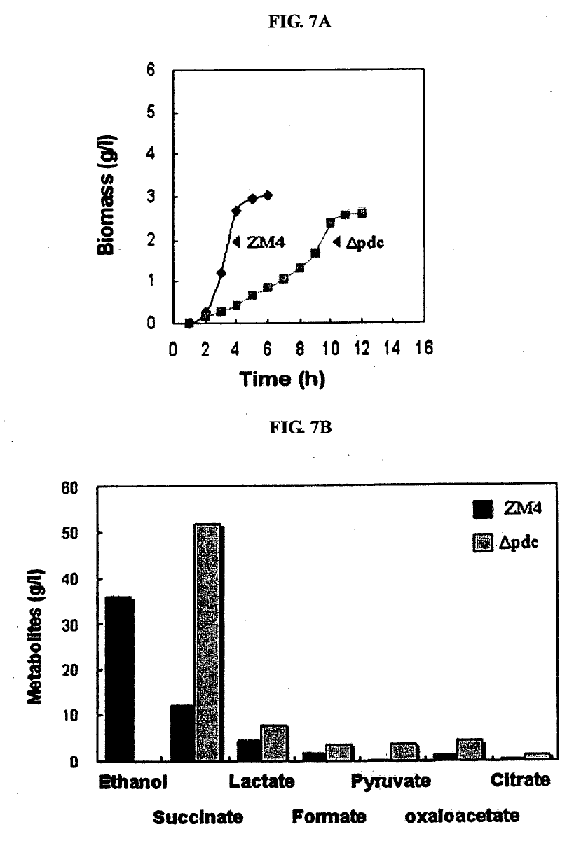

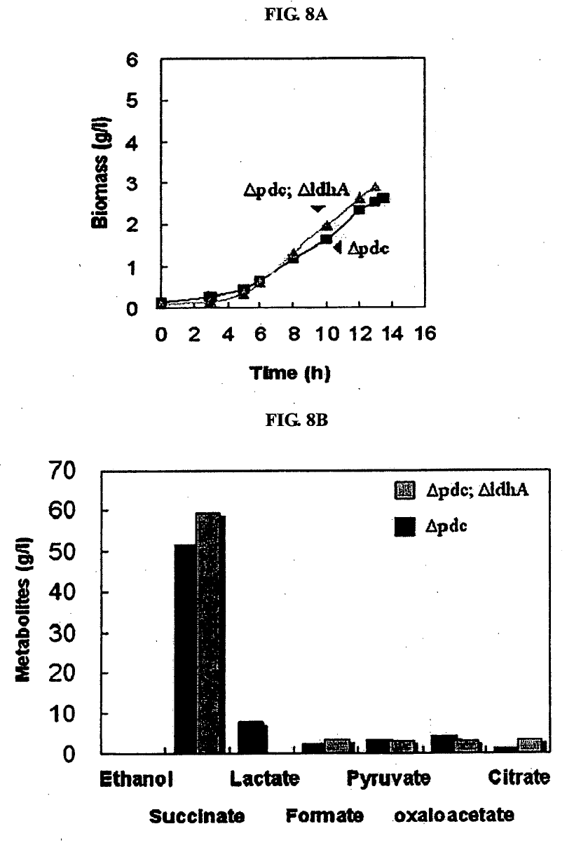

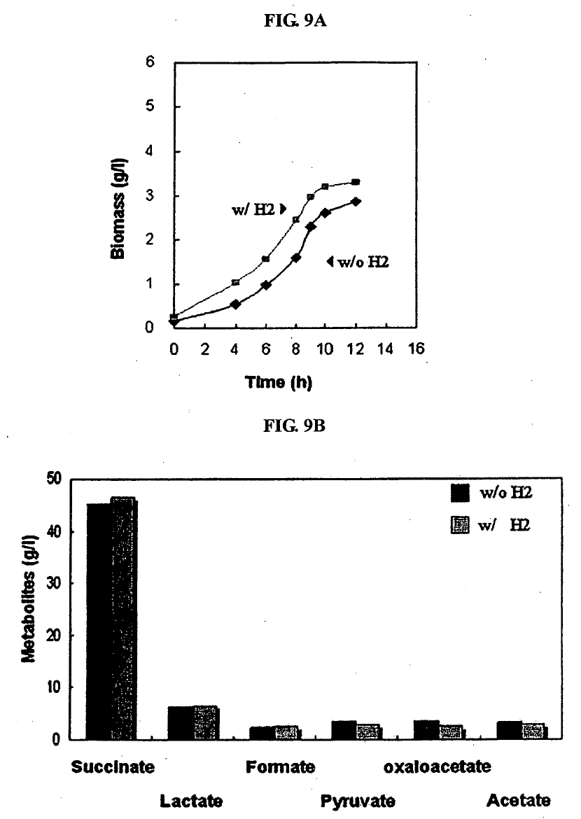

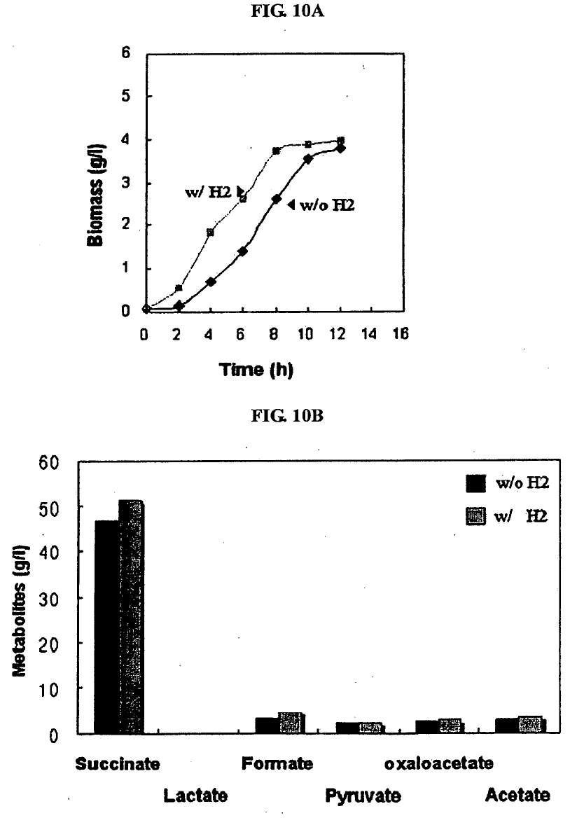

The process was repeated 3 times, and the average of the results is shown in the following Table 2, and FIGS. 7A to 10B, respectively.

TABLE 2

| Z. | | | | | | | | succinate |
|---|---|---|---|---|---|---|---|---|
| mobilis | glucose | ethanol | succinate | lactate | formate | acetate | yield | molar |
| strain | (g/l) | (g/l) | (g/l) | (g/l) | (g/l) | (g/l) | (%) | yield |
| ZM4 | 100.0 | 46.20 | 9.60 | 6.40 | — | 1.20 | 10 | 0.15 |
| ΔldhA | 106.88 | 58.01 | 17.82 | — | 3.32 | 3.13 | 17 | 0.25 |
| *Δpdc | 62.30 | — | 56.46 | 8.54 | 3.43 | 3.86 | 90 | 1.38 |
| *Δpdc/ | 67.02 | 0.00 | 63.93 | — | 2.15 | >2.0 | 95 | 1.46 |
| ΔldhA- | | | | | | | | |
| NaHCO₃ | | | | | | | | |
| **Δpdc/ | 55.56 | 0.00 | 51.95 | — | 3.57 | 3.39 | 94 | 1.43 |
| ΔldhA- | | | | | | | | |
| Gas | | | | | | | | |

*culture in a RM medium containing 10 mM of NaHCO₃
**culture in a RM medium containing carbon dioxide-hydrogen mixing gas (mixing ratio = 1:1) (1 vvm)

As shown in the Table 2, the transformants prepared from Examples 1 to 3 were confirmed with increased succinate productivity compared to the wild-type.

Also, the ΔldhA::cm^{R} transformant was confirmed with excellent ethanol productivity, and the Δpdc::tet^{R} transformant was confirmed with excellent succinate and lactate productivity, respectively.

### Example 5

### Test for Cell Growth Rate, and for Productivity of Primary Metabolites

The transformants were cultured with identical methods to Example 4, and kinetic analysis was evaluated to utilize as a measure for determining biomass growth and primary metabolite production (hereinafter referred to as ‘product’) according to time. In the kinetic analysis, the values measured in an exponential growth phase, namely a point between maximum biomass growth and product, were obtained by the following method:

1. Specific growth rate (μₘₐₓ) (h⁻¹)

dx=μ×dt (μ=specific growth rate) <Formula 1.1>

μ=1/t×ln(X/X₀) <Formula 1.2>

t=time (h);

X=biomass (g);

dx=biomass difference;

dt=time difference.

2. Biomass yield (Yx/s) and product yield (Yp/s)

Yx/s=−dx/ds <Formula 1.3>

Yx/s=(X−X₀)/(S₀−S) <Formula 1.4>

dx=biomass difference;

ds=substrate (glucose) difference.

Yp/s=dp/ds <Formula 1.5>

Yp/s=(P−P₀)/(S₀−S) <Formula 1.6>

dp=product difference;

ds=substrate (glucose) difference.

3. Specific glucose consumption rate (qₛ) (gg⁻¹h⁻¹)

ds=−qₛ×dt (qs=specific glucose consumption rate) <Formula 1.7>

From Formulas 1.1 and 1.3,

qₛ=(1/Yx/s)×μ <Formula 1.8>

4. Specific succinate production rate (qₚ)(gg⁻¹ h⁻¹)

dp=qₚ×dt (qs=specific succinate production rate) <Formula 1.9>

From Formulas 1.1 and 1.4,

qₚ=(Yp/x)×μ <Formula 1.10>

5. Productivity (gl⁻¹h⁻¹)

Because a kinetic parameter of the productivity does not exist, maximum product concentration produced during the exponential growth phase which is the most active production period, was depicted with the following method:

P (Productivity)=dP/dt <Formula 1.11>

dP=product difference during exponential growth phase (gl⁻¹)

dt=time difference during exponential growth phase (h)

Also, rough values can be calculated with the following method:

P=(Yp/s)/(Yx/s)×μ <Formula 1.12>

6. Succinic acid molar yield

The succinic acid molar yield has an identical meaning as the product yield, wherein the former was expressed as a molar yield not a percentage (%). Theoretically, because succinic acid produced from 1 mole (180 g) of glucose is only 2 moles (236 g), after make changing actually produced succinic acid (g) with mole concentration, the value divides with the value make changing glucose (g) used in the experiment with mole concentration. As a result, the purpose value was obtained.

[succinic acid (g)/glucose (g)] <Formula 1.13>

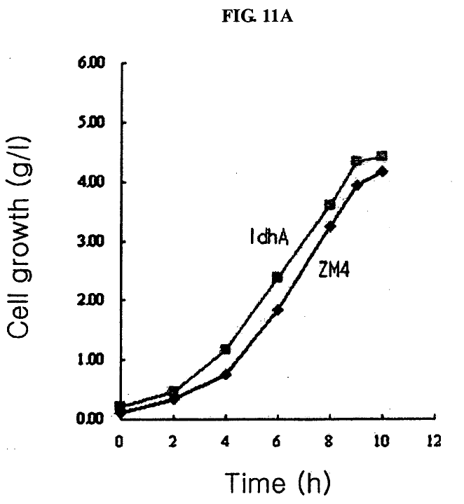

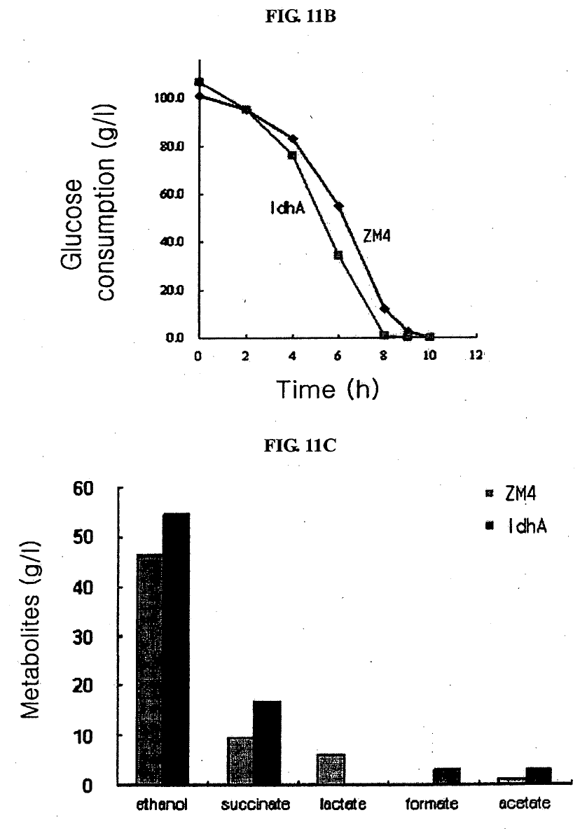

The results of kinetic analysis toward a wild-type ZM4, a Δpdc transformant, and a Δpdc/ΔldhA transformant are depicted in the following Table 3, and the cell growth, the glucose consumption, and the product yield are depicted in FIGS. 11A to 11C, respectively.

TABLE 3

| | ZM4 | | pdc | | pdc-ldhA | |
|---|---|---|---|---|---|---|
| Kinetic parameters | −H_{g} | +H_{g} | −H_{g} | +H_{g} | −H_{g} | +H_{g} |
| Specific growth rate, | 0.3 | 0.45 | 0.11 | 0.2 | 0.15 | 0.25 |
| μ max (h⁻¹) | | | | | | |
| Specific glucose | 5.2 | 5.95 | 3.07 | 4.65 | 4.04 | 5.26 |
| consumption rate, | | | | | | |
| qs (g g⁻¹ h⁻¹) | | | | | | |
| Specific succinate | 1.43 | 1.56 | 2.75 | 3.12 | 3.46 | 3.86 |
| production rate, | | | | | | |
| qp (g g⁻¹ h⁻¹) | | | | | | |
| Biomass yield (Yx/s) | 0.06 | 0.06 | 0.03 | 0.05 | 0.04 | 0.07 |
| Product yield (Yp/s) | 0.28 | 0.26 | 0.9 | 0.93 | 0.95 | 1.02 |
| Productivity (g l⁻¹ h⁻¹) | 1.2 | 1.6 | 3.8 | 5.1 | 5.3 | 5.9 |
| Succinic acid molar yield | 0.23 | 0.48 | 1.38 | 1.42 | 1.46 | 1.55 |

The present invention provides a method for mass production of various primary metabolites containing organic acids that have environmental friendly and industrial applicability by inhibiting specific a metabolite of metabolism in microorganisms, and the organic acids according to the present invention can be used instead of previous chemical synthesis materials in various fields, and it can also can provide the effects of expense reduction and environmental protection.

1. A method for mass-producing at least one primary metabolite selected from the group consisting of ethanol, lactate, pyruvate, citrate, glutamate, succinate, fumarate, and malate by a Zymomonas mobilis (Z. mobilis) strain, by deleting at least one gene selected from the group consisting of a pyruvate decarboxylase coding pdc gene (SEQ ID NO: 1) and a lactate dehydrogenase coding ldhA gene (SEQ ID NO: 2) from a Z. mobilis genome.

2. The method for mass-producing a primary metabolite according to claim 1, wherein at least one primary metabolite selected from the group consisting of succinate and lactate is produced by deleting the pdc gene (SEQ ID NO: 1).

3. The method for mass-producing primary a metabolite according to claim 1, wherein at least one primary metabolite selected from the group consisting of ethanol and succinate is produced by deleting the ldhA gene (SEQ ID NO: 2).

4. The method for mass-producing succinate according to claim 1, wherein the succinate is produced by deleting both of the pdc gene (SEQ ID NO: 1) and the ldhA gene (SEQ ID NO: 2).

5. A transformant for mass-producing at least one primary metabolite selected from the group consisting of ethanol, lactate, pyruvate, citrate, glutamate, succinate, fumarate, and malate, wherein the transformant is prepared by deleting at least one gene selected from the group consisting of a pdc gene (SEQ ID NO: 1) and a ldhA gene (SEQ ID NO: 2) from Zymomonas mobilis genome.

6. The transformant according to claim 5, wherein the pdc gene (SEQ ID NO: 1) is deleted from Z. mobilis, thereby increasing the production of at least one primary metabolite selected from the group consisting of succinate and lactate.

7. The transformant according to claim 5, wherein the ldhA gene (SEQ ID NO: 2) is deleted from Z. mobilis, thereby increasing the production of at least one primary metabolite selected from the group consisting of ethanol and succinate.

8. The transformant according to claim 5, wherein both of the pdc gene (SEQ ID NO: 1) and the ldhA gene (SEQ ID NO: 2) are deleted from Z. mobilis, thereby increasing the production of succinate.

9. The transformant according to claim 5, wherein the transformant is a strain selected from the group consisting of KCTC 11012BP, KCTC 1113BP, and KCTC 10908BP.

10. A method of preparing a Z. mobilis transformant according to claim 5, comprising deleting at least one gene selected from the group consisting of a pdc gene (SEQ ID NO: 1) and a ldhA gene (SEQ ID NO: 2) from a Z. mobilis genome.

11. The method according to claim 10, further comprising:
cloning the fragment containing the Z. mobilis pdc gene (SEQ ID NO: 1) into a plasmid;
removing the pdc gene from the pdc gene-containing plasmid; and
transforming the pdc gene-deleted plasmid into a Z. mobilis genome.

12. The method according to claim 11, wherein the fragment containing the pdc gene comprises 1,500 to 5,000 bp of a homologous region for homologous recombination located in both of the 5′- and 3′-terminal regions of the pdc gene together with the Z. mobilis pdc gene.

13. The method according to claim 10, further comprising:
cloning the fragment containing the Z. mobilis ldhA gene (SEQ ID NO: 2) into a plasmid;
removing the ldhA gene from the ldhA gene-containing plasmid; and
transforming the ldhA gene-deleted plasmid into a Z. mobilis genome.

14. The method according to claim 13, wherein the fragment containing the ldhA gene comprises 1,500 to 5,000 bp of a homologous region for homologous recombination located in both of the 5′- and 3′-terminal regions of the ldhA gene together with the Z. mobilis ldhA gene.

15. The method according to claim 10, further comprising consecutive steps of:
cloning the fragment containing the Z. mobilis pdc gene (SEQ ID NO: 1) into a plasmid;
removing the pdc gene from the pdc gene-containing plasmid;
transforming the pdc gene-deleted plasmid into a Z. mobilis genome; and
cloning the fragment containing the Z. mobilis ldhA gene (SEQ ID NO: 2) into a plasmid;
removing ldhA gene from the ldhA gene-containing plasmid;
transforming the ldhA gene-deleted plasmid into a Z. mobilis genome.

16. A method for mass-producing at least one primary metabolite selected from the group consisting of ethanol, lactate, pyruvate, citrate, glutamate, succinate, fumarate, and malate, comprising the steps of:
preparing a Z. mobilis transformant where at least one gene selected from the group consisting of pdc gene (SEQ ID NO: 1) and ldhA gene (SEQ ID NO: 2) is deleted; and
culturing the Z. mobilis transformant for 10 to 14 h at 30 to 34° C.

17. The method for mass-producing a primary metabolite according to claim 16, wherein the step of culturing the Z. mobilis transformant is performed by adding 0.2 to 1 vvm of carbon dioxide gas, or using a culture medium containing 1 to 50 mM of carbonate.

18. The method for mass-producing a primary metabolite according to claim 17, wherein the carbonate is selected from the group consisting of NAHCO₃, NA₂CO₃, and CaCO₃.

19. The method for mass-producing a primary metabolite according to claim 16, wherein the step of culturing the Z. mobilis transformant is performed by additionally adding 0.2 to 1 vvm of hydrogen gas.
